(19) United States
(12) Patent Application Publication
(10) Pub. No.: US 2022/0193347 A1
(43) Pub. Date: Jun. 23, 2022

(54) ADMINISTRATION DEVICE FOR ADMINISTRATION OF A FLUID

(71) Applicant: Boehringer Ingelheim International GmbH, Ingelheim am Rhein (DE)

(72) Inventors: Axel ROTH, Ingelheim am Rhein (DE); Viola K. HOLMAN, St. Paul, MN (US); Hans Jøergen JENSEN, Holstebro (DK)

(73) Assignee: Boehringer Ingelheim International GmbH, Ingelheim am Rhein (DE)

(21) Appl. No.: 17/604,033

(22) PCT Filed: Apr. 16, 2020
(86) PCT No.: PCT/EP2020/060690

§ 371 Date: Oct. 15, 2021

(30) Foreign Application Priority Data
Apr. 18, 2019 (EP) 19170230.7

(51) Int. Cl. A61M 5/31; A61M 5/315; A61M 5/32

(52) U.S. Cl. CPC A61M 5/3146; A61M 5/3135; A61M 5/31511; A61M 5/31501; A61M 5/3158; A61M 5/31535; A61M 2210/0612; A61M 5/3204

(57) ABSTRACT

An administration device for in particular intravitreal administration of a fluid, the administration device having a preferably pre-filled syringe and an actuation mechanism for actuating the syringe. The administration device preferably includes a priming device, in particular in addition to the actuation mechanism. It is preferably prevented that the priming device be advanced beyond a defined end position. The syringe can preferably be moved within a housing of the administration device and/or positioned completely inside the housing.

### BACKGROUND OF THE INVENTION

### Field of the Invention

The present invention relates to an administration device for in particular intravitreal administration of a fluid as well as to a use of an administration device.

Although the present invention is in particular suitable for intravitreal injection of a drug, i.e., injection of a drug directly into the eye, in particular the vitreous body, the administration device of the present invention can also be used in other contexts, in particular for administration or injection of a fluid or a drug. The present invention is particularly advantageous in the medical field for administrating or injecting a drug or other fluid directly into a human or animal body or a part thereof.

### Description of the Related Art

International Patent Application Publication WO 2014/005728 A1 and corresponding U.S. Pat. No. 9,220,631 B2 relate to a small volume syringe suitable for ophthalmic injections. The syringe is pre-filled with a drug and comprises a body, a stopper and a plunger. The plunger comprises a plunger contact surface arranged to contact the stopper such that the plunger can be used to force the stopper towards an outlet and of the syringe body.

International Patent Application Publication WO 00/69488 A2 and corresponding U.S. Pat. No. 6,599,272 B1 relate to an injector device comprising a housing and a syringe with a needle, the syringe being movable relative to the housing. The injector device comprises a penetration arrangement operable to move the needle from a rear position to a forward position, a return arrangement to move the needle in a rearward direction, and an injection arrangement to expel content of the syringe through the needle. Further, the injection device comprises a control button arranged on the housing, which stepwise triggers the penetration arrangement and injection arrangement. The button is arranged to have at least a movement component perpendicular to the needle axis. The injector device is not adapted for intravitreal injection.

U.S. Patent Application Publication 2006/0069350 A1 relates to a medical syringe injector pen with a housing for accommodating a syringe. The pen comprises a drive mechanism and an actuation member for releasing the drive mechanism and actuating the device to deliver a fluid from the syringe to a user. After delivering the fluid, the syringe is retracted back into the housing. The injector pen is not adapted for intravitreal injection.

U.S. Patent Application Publication 2010/0241102 A1 relates to an intravitreal injection device including an injection assembly adapted to receive a syringe. The injection assembly, in response to a user input, is adapted to automatically and sequentially first, transfer the syringe into the patient's eye and then second, dispense the injectant from the syringe. The injection device does not have a priming device.

UK Patent Application Publication GB 2541445 A and corresponding U.S. Pat. No. 10,512,729 B2 relate to an injection device. The injection device contains a pre-filled syringe with a stopper for expelling medicament. A housing contains the syringe, a plunger for driving the stopper, and a priming mechanism having an operating member which is movable with respect to the housing. The priming mechanism is arranged to move the syringe towards the plunger upon movement of the operating member, so that the stopper comes into contact with the plunger. Rotation of the operating member causes a shuttle to be axially displaced so it presses on the shoulder of the syringe barrel. The priming is driven by removing a cap from the injector. The injection device is not adapted for intravitreal injection.

### SUMMARY OF THE INVENTION

It is an object of the present invention to provide an administration device which is safe to use, easy to use and/or ensures a correct and/or exact dosing of a fluid or drug to be administered, in particular in the case of intravitreal administration.

The above object is solved by an administration device and method as described herein below.

An administration device according to the invention comprises a syringe and an actuation mechanism for actuating the syringe. The syringe is preferably a pre-filled syringe, i.e., a syringe containing a fluid or drug to be dispensed from the syringe and/or administered to a patient. The syringe comprises a syringe body and a piston arranged within the body, the piston being moveable within the syringe body.

According to one aspect of the present invention, the administration device comprises a housing, wherein the actuation mechanism is configured for moving the syringe or the syringe body relative to the housing. This is beneficial for an easy use and handling of the administration device.

According to a further aspect which can also be implemented independently, the administration device comprises a housing, wherein the syringe and/or an injection needle attached thereto can be or is/are configured to be positioned completely inside the housing, in particular so that the syringe and/or injection needle do not jut out of the housing. This is conducive to a safe handling of the administration device.

According to a further aspect which can also be implemented independently, the administration device comprises a priming device in addition to the actuation mechanism. In particular, the priming device is separated and/or functionally decoupled from the actuation mechanism. In this way, priming can be performed without the possibility of accidentally actuating the syringe which could lead to an unintended dispensing of fluid. Thus, easy and safe handling of the administration device can be achieved.

According to a further aspect which can also be implemented independently, the administration device comprises a priming device configured to act on a piston of the syringe and a blocking device for preventing the priming device from moving the piston beyond a priming position and/or to an end of the syringe. This ensures a correct, exact and/or defined priming and is conducive to an easy and safe handling of the administration device. In particular, the blocking device is configured to prevent the priming device from being advanced beyond the defined end position.

The syringe preferably comprises or is connectable to an injection needle, in particular by a Luer connection.

The actuation mechanism is preferably configured to move the syringe, in particular the syringe body, relative to the housing into an administration position of the syringe. In particular, the syringe or the piston thereof is not actuated while the syringe is being moved into the administration position. This is conducive to a safe and easy handling of the administration device.

Further, the actuation mechanism is preferably configured to actuate the syringe subsequent to moving the syringe, in particular the syringe body into the administration position. The steps of first moving the syringe or syringe body into the administration position and subsequently actuating the syringe are preferably performed in a common movement of the actuation mechanism. In particular, the syringe is actuated by moving the piston located in the body of the syringe. This conducive to a safe and easy handling of the administration device.

Further, the actuation mechanism is preferably configured to, in particular automatically, position the syringe and/or injection needle completely inside the housing subsequent to actuating the syringe. In other words, the syringe and/or injection needle are, preferably automatically, retracted into or covered by the housing after actuating the syringe, in particular after administration or dispensing of the fluid. Thus, a safe and easy handling of the administration device is achieved.

It is preferred that the actuation mechanism comprises a return means, in particular a spring, for moving the syringe and the housing or a part thereof relative to each other. This is conducive to a safe and easy handling of the administration device.

According to a preferred aspect, the actuation mechanism comprises a pushing element for actuating or moving the piston of the syringe, the pushing element preferably comprising a coupling device for coupling to the syringe body, in particular in an administration position, particularly so that the position of the syringe or syringe body relative to the pushing element is fixed by the coupling. This is conducive to a safe and easy handling of the administration device.

In a preferred embodiment, the actuation mechanism comprises a preferably button-like actuation element. The actuation element is preferably configured for manual and/or direct actuation by a user of the administration device, in particular independently of an actuation of the priming device. Preferably, the actuation element is configured to be actuated in a direction transverse, in particular perpendicular to a longitudinal extension of the syringe and/or to a direction of movement of the priming device. This is conducive to a safe and easy handling of the administration device.

The actuation mechanism preferably comprises a transmission or gear for moving the syringe. It is preferred that the transmission is driven by the button-like actuation element. This is conducive to a safe and easy handling of the administration device.

The actuation element is preferably arranged laterally at the housing and/or at or close to an axial end of the administration device comprising an administration opening and/or close to an axial end of the administration device which is opposite to an axial end comprising the priming device or an actuation section thereof. In this way, the administration is preferably designed for one-handed actuation and/or can be actuated by an index finger of a user, in particular while holding the administration device like a pen.

According to a preferred aspect, the syringe and/or the injection needle can be retracted, preferably automatically, into the housing, in particular by the transmission. Retracting the syringe and/or injection needle is preferably performed after administration or dispensing of the fluid, in particular automatically. This is conducive to a safe and easy handling of the administration device.

According to another preferred embodiment, the housing comprises or consists of two housing parts which are moveable relative to each other and/or relative to the syringe. This is conducive to a safe and easy handling of the administration device.

Preferably, the above-mentioned return means or spring is arranged within the housing and/or between the two housing parts. Preferably, the spring is configured to move the housing parts apart. In this way, automatically moving the housing parts apart can be achieved, for example, for covering the syringe and/or injection needle by one of the housing parts or retracting the syringe into the housing, in particular after administrating or dispensing a fluid. This is conducive to a safe and easy handling of the administration device.

According to a further preferred aspect, the administration device is configured in such a way that during actuation of the piston of the syringe, a first one of the two housing parts moves relative to the syringe body and a second one of the two housing parts, wherein the syringe body and the first housing part are not moved relative to each other. Particularly, a pushing element for moving the piston is rigidly connected to the first housing part or forms a part or section of the first housing part. This is conducive to a safe and easy handling of the administration device.

The term “priming” in the sense of the present invention preferably means a preparation of the administration device prior to administration of a fluid contained in the administration device or the syringe, wherein a small part of the fluid contained in the syringe is dispensed so that air bubbles potentially contained in the fluid are removed and the volume contained in the syringe after priming corresponds to a desired dose to be administered to a patient. Typically, the volume of the fluid contained in the (pre-filled) syringe exceeds the volume of the dose to be administered a little, so that air contained in the syringe can be removed by priming without affecting the dose to be administered. However, the use of the term “priming” does not imply that the syringe is actually filled with a fluid. In other words, “priming” in particular means a movement of a piston of the syringe into a defined position, hereinafter also referred to as “priming position”. In particular, the priming device can be actuated or moved even if the syringe is empty (i.e., not filled with a liquid or drug). Accordingly, all other actions performed during or for priming, such as a movement of the priming device or a movement of the piston of the syringe, can be performed with an empty syringe, i.e., a syringe which is not pre-filled.

The term “administration position” in the sense of the present invention preferably means a position of the syringe and/or an injection needle attached thereto in which a fluid contained in the syringe can be dispensed or administered to a patient without performing further steps apart from actuation of the syringe for dispensing the fluid. In particular, the administration position is the position of the syringe body during administration of fluid contained in the syringe to a patient. However, the administration position is defined with respect to other parts of the administration device, in particular the housing or parts thereof. Thus, the administration position does not refer to a patient.

The terms “administer” and “dispense” as well as terms related thereto in the sense of the present invention preferably mean dispensing a fluid from the administration device or syringe, in particular through an injection needle attached to the syringe. While it is preferred that the administration device according to the invention is used for administering a drug directly to the body of a patient, for example by using an injection needle and/or by injecting the drug directly into the eye, in particular the vitreous body, these terms also refer to dispensing a fluid without a patient being present. In particular, the term “administer” can also be used for dispensing a fluid without the presence of a patient.

The term “actuating the syringe” and related terms in the sense of the present invention preferably mean a movement of the piston relative to the syringe body, in particular in a direction towards an outlet of the syringe through which fluid can be dispensed. Thus, the term “actuating the syringe” can also be replaced by the term “actuating the piston”.

The term “moving the syringe” in the sense of the present invention preferably means a preferably linear movement of the syringe body, in particular relative to other parts of the administration device, for example the housing. In particular, only actuating or moving the piston relative to the syringe body without moving the syringe body is not understood as a movement of the syringe in the sense of the present invention. Particularly, the term “moving the syringe” can thus also be replaced by the term “moving the piston”. Further, the term “syringe” can be replaced by the term “syringe body” when it is readily apparent from the context that the term “syringe” in particular refers to the syringe body, such as in the term “moving the syringe”.

The above-mentioned aspects and features of the present invention and the aspects and features of the present invention that will become apparent from the claims and the following description can in principle be implemented independently from one another, but also in any combination or order.

Further aspects, advantages, features and properties of the present invention will become apparent from the following description of preferred embodiments in conjunction with the accompanying drawings.

### BRIEF DESCRIPTION OF THE DRAWINGS

FIG. 1 shows a schematic, perspective view of a first embodiment of an administration device according to the invention;

FIG. 2 shows a schematic side view of the administration device according to the first embodiment being held by a user;

FIG. 3 shows a schematic section of the administration device according to the first embodiment, wherein the syringe is in a rest position;

FIG. 4 shows a schematic section of the administration device according to the first embodiment, wherein priming has been performed;

FIG. 5 shows a schematic section of the administration device according to the first embodiment, wherein the syringe is in an administration position and the syringe is not actuated;

FIG. 6 shows a schematic section of the administration device according to the first embodiment, wherein the syringe is in an administration position and the syringe is actuated;

FIG. 7 shows a schematic section of the administration device according to the first embodiment, wherein the syringe is positioned in a retracted or rest position;

FIG. 8 shows a schematic, perspective view of a second embodiment of an administration device according to the invention;

FIG. 9 shows a schematic side view of the administration device according to the second embodiment being held by a user;

FIG. 10 shows a schematic section of the administration device according to the second embodiment, wherein the syringe is in a rest position;

FIG. 11 shows a schematic section of the administration device according to the second embodiment, wherein the syringe is in an administration position and the syringe is not actuated;

FIG. 12 shows a schematic section of the administration device according to the second embodiment, wherein syringe is actuated;

FIG. 13 shows a schematic section of the administration device according to the second embodiment, wherein the syringe is positioned within a housing of the administration device;

FIG. 14 shows a schematic, perspective view of the administration device according to a third embodiment; and

FIG. 15 shows a schematic section of the administration device according to the third embodiment.

### DETAILED DESCRIPTION OF THE INVENTION

In the figures, which are only schematic and sometimes not to scale, the same reference signs are used for the same or similar parts and components, wherein corresponding or comparable properties and advantages can be achieved even if these are not repeatedly described.

The figures show different embodiments of an administration device according to the invention, wherein the embodiments shown in particular have several common features. Thus, features described in connection with one embodiment can preferably also be realized with another embodiment. Thus, for the sake of conciseness, repeated description of features common to more than one embodiment is avoided. Further, different embodiments can in particular be combined.

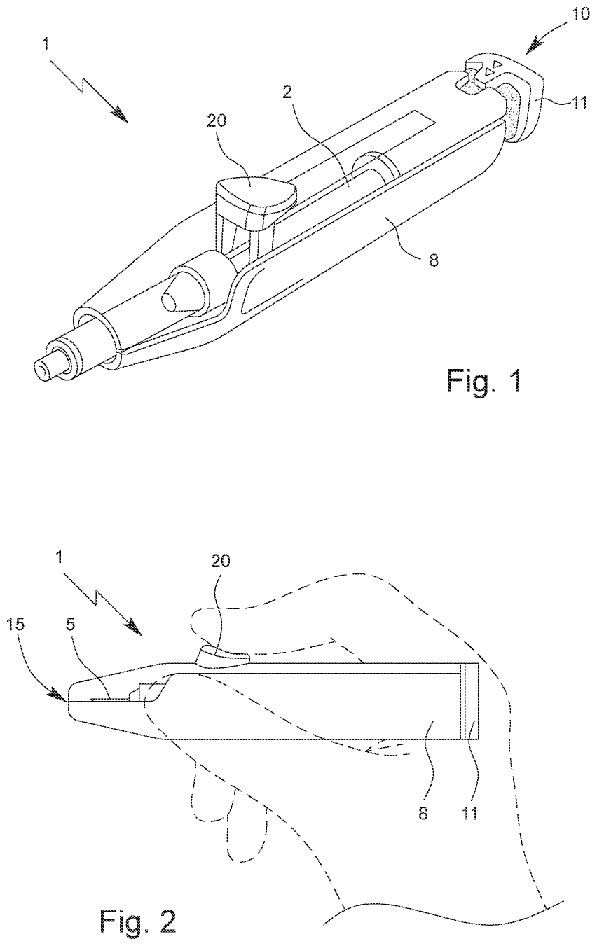

In the following, the present invention will first be described with reference to FIGS. 1 to 6, which show a first preferred embodiment of the present invention in several depictions. Thereafter, further embodiments will be described.

FIG. 1 shows an administration device 1 according to the first embodiment in a perspective view.

The administration device 1 is in particular an intravitreal administration device and/or configured for intravitreal administration and/or injection of a fluid F, in particular a medicament, i.e., administration and/or injection of the fluid F directly into an eye E, in particular the vitreous body thereof, of a patient.

Preferably, all of the parts of the administration device 1 described in the following are sterilized and/or can be sterilized. In particular, the administration device and/or its parts are configured to be sterilized by an autoclave.

The administration device 1 comprises a syringe 2. The administration device 1 or syringe 2 is configured to dispense a fluid F, in particular to administer the fluid F directly to a body of a patient (not shown) or a part thereof, in particular an eye E, particularly preferred the vitreous body. This is conducive to a safe and easy handling of the administration device.

The syringe 2 is preferably pre-installed and/or permanently installed in and/or integral with the administration device 1. However, solutions in which the syringe 2 can be inserted into the administration device 1 by a user are also conceivable.

The administration device 1 and/or syringe 2 is preferably a disposable article and/or configured for single use.

The syringe 2 comprises or consists of a syringe body 3 and a piston 4.

The syringe body 3 is preferably at least essentially cylindrical and/or elongated, in particular designed barrel-like. In particular, the syringe 2 or syringe body 3 are arranged at least essentially parallel, in particular coaxial and/or symmetric, to a longitudinal extension or axis L of the administration device 1 and/or syringe 2.

Terms as “axial”, “coaxial”, “radial” or the like preferably relate to the axis L if not explicitly indicated otherwise.

The piston 4 is arranged within the body 3 and preferably fits tightly and/or sealingly in the body 3 so that fluid F cannot pass between the piston 4 and the body 3.

The piston 4 is moveable within the syringe body 3, in particular linearly and/or along the axis L.

The syringe body 3 preferably comprises a collar 3A at the axial end opposite the outlet 7. In particular, the collar 3A surrounds the cylindrical section of the syringe body 3, in particular in an annular fashion, and/or protrudes radially from the syringe body 3.

According to a preferred aspect, the syringe 2 is pre-filled with or contains the fluid F. The fluid F is in particular a drug or medicament to be administered to a patient, for example a VEGF-Antagonist, Bevacizumab, Ranibizumab, Aflibercept and/or Pegaptanib. However, other drugs or medicaments are also possible.

Further, it is also possible that the syringe 2 is configured to be filled or re-filled by a user of the administration device 1 and/or that the syringe 2 is not pre-filled. As an alternative or in addition, the syringe 2 can be taken out of the housing 8 and/or be exchanged.

The syringe 2 can also comprise an injection needle 5, for example a staked needle and/or a needle which is not detachable from the syringe 2. However, it is also possible that the syringe 2 is merely configured to be connected to an injection needle 5 and/or that the injection needle 5 can be detached from the syringe 2, in particular by a Luer connection or system which is widely used in the medical field, in particular a Luer lock or Luer slip.

The syringe 2 comprises or defines a chamber 6 for the fluid F. The chamber 6 is preferably formed or delimited by the body 3 and the piston 4. The chamber 6 has a variable volume. The volume of the chamber 6 can be changed, in particular reduced, by moving the piston 4 relative to the syringe body 3.

Further, the syringe 2 or syringe body 3 comprises an outlet 7 for dispensing the fluid F. The outlet 7 is preferably arranged at an axial end of the syringe 2.

The piston 4 is preferably only moveable towards the outlet 7 and/or not moveable away from the outlet 7.

The injection needle 5 is preferably arranged at or can be connected to the outlet 7.

The fluid F can be dispensed from the syringe 2 by moving the piston 4 towards the outlet 7, so that the volume of the chamber 6 is reduced and fluid F contained in the chamber 6 is expelled through the outlet 7.

The administration device 1 preferably comprises a housing 8. The syringe 2 is preferably arranged inside the housing 8. The housing 8 preferably surrounds or encloses the syringe 2 or at least a major part of the syringe body 3. Preferably, the syringe 2 can be completely positioned inside the housing 8, so that no part of the syringe 2 juts out of the housing 8. In particular, this also holds for an injection needle 5 being attached to or forming a part of the syringe 2.

The housing 8 can be at least partially transparent so that visual inspection of the inside of the administration device 1, in particular of the syringe 2, is allowed.

The housing 8 preferably protects the syringe 2, the syringe body 3 and/or the needle 5 against external influences.

The administration device 1 preferably comprises an in particular rigid cover or shield 27 for the injection needle 5. In particular, the shield 27 is configured to protect the injection needle 5 against damages and/or to ensure sterility of the injection needle 5.

The shield 27 is preferably detachable from the injection needle 5, administration device 1, housing 8 and/or syringe 2.

The housing 8 preferably comprises an administration opening 15 for the syringe 2 and/or injection needle 5 at an axial end of the administration device 1.

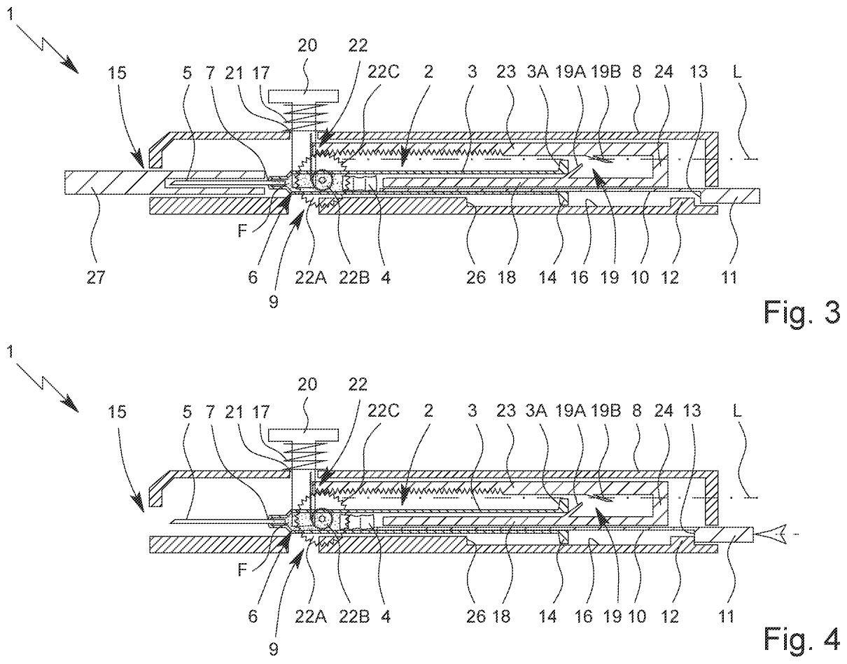

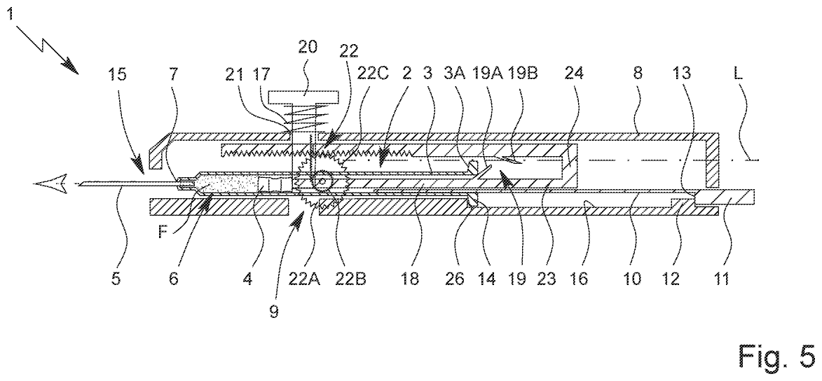

The syringe 2 or syringe body 3 is preferably moveable within and/or relative to the housing 8. In particular, the syringe 2 or syringe body 3 is moveable between a retracted or rest position, which is in particular shown in FIGS. 3 and 4, and an administration position, which is in particular shown in FIG. 5. The retracted or rest position is in particular the position in which the syringe 2 is positioned when the administration device 1 is not used or actuated.

The administration position is in particular a position in which dispensing of the fluid F for administration of the fluid F to a patient can take place. In the administration position, the outlet 7 and/or injection needle 5 preferably juts out of the housing 8. In the administration position, the injection needle 5 and/or the syringe 2, in particular the outlet 7, preferably extends through the administration opening 15.

The administration device 1 comprises an actuation mechanism 9 for actuating the syringe 2, in particular for moving the piston 4 relative to the syringe body 3, particularly preferably towards the outlet 7.

Preferably, the administration device 1 comprises, in particular in addition to and/or separately from the actuation mechanism 9, a priming device 10 configured for priming. The term “priming” is defined above.

The priming device 10 is preferably separate and/or functionally decoupled from the actuation mechanism 9. Thus, priming, in particular by the priming device 10, can be performed without actuating the syringe 2 by the actuation mechanism 9.

The priming device 10 is preferably configured and/or arranged to actuate the piston 4 and/or to move the piston 4 relative to the syringe body 3. In particular, the priming device 10 is configured to move or push the piston 4 towards the outlet 7 of the syringe 2.

The priming device 10 is preferably configured to move the piston 4 into a defined end position, hereinafter also referred to as “priming position”. In particular, the piston 4 is not moveable beyond the priming position by the priming device 10.

The term “priming position” preferably means a position into which the piston 4 is moved for priming. When the piston 4 is in the priming position, the chamber 6 preferably has (exactly) a desired or predetermined volume and/or contains an amount of fluid F corresponding to said volume, in particular a volume corresponding exactly to one dose of the fluid F which is to be administered to a patient.

In the priming position, the piston 4 is preferably spaced apart from the axial end of the syringe 2 comprising the outlet 7.

The administration device 1 and/or priming device 10 is preferably configured in such a way that actuation of the administration device 1, in particular dispensing a drug and/or moving the piston 4 beyond the priming position, is only possible after priming and/or prevented before and/or during priming. For example, the priming device 10 can be configured to (mechanically) block actuation of the administration device 1 before being moved into the priming position.

The syringe body 3 is preferably open at an axial end 14 of the syringe 2 or syringe body 3 opposite the outlet 7. In particular, the priming device 10 extends into the syringe body 3 through the axial end 14 or the opening thereof.

The priming device 10 is preferably moveable relative to the housing 8 and/or relative to the syringe 2, in particular along and/or parallel to the axis L.

The priming device 10 can be configured for unique actuation. In other words, it is possible that the priming device 10 can be actuated only once. The priming device 10 is preferably configured to be locked upon moving the piston 4 into the priming position by the priming device 10, in particular by engaging with the housing 8 or a part thereof. Preferably, the priming device 10 is not moveable again after priming.

Locking the priming device 10 by or after priming preferably serves as an indication to a user of the administration device 1 that priming has already been performed, that the administration device 1 is ready for use, in particular administration of a drug to a patient, and/or that the administration device 1 has already been used.

As an alternative or in addition, the priming device 10 and/or administration device 1 can comprise a cap or the like for covering the priming device 10 (not shown). In particular, the cap can be detachable from the housing 8 or administration device 1. The cap preferably covers the priming device 10 so that an (accidental) actuation of the priming device 10 is prevented by the cap.

The priming device 10 is preferably physically isolated from the piston 4. Thus, it is preferably not possible to move the piston 4 in a direction away from the outlet 7 by the priming device 10. This prevents the piston 4 from being moved away from the outlet 7, for example, when the priming device 10 is (accidentally) moved away from the piston 4 and/or outlet 7. Thus, it is prevented that air be drawn into the syringe 2 and/or the piston 4 be moved away from the outlet 7 while fluid F is administered or injected into the body of a patient. Harming the patient by accidentally moving the priming device 10 in the wrong direction is thus avoided.

The priming device 10 is preferably arranged partly within the housing 8 and/or the syringe body 3 and/or extends into the syringe 2 or syringe body 3.

Preferably, the priming device 10 comprises an actuation section 11 which is in particular arranged outside the housing 8 so that the actuation section 11 can be actuated by a user. Preferably, the actuation section 11 is arranged on an axial end of the administration device 1, in particular opposite the administration opening 15.

The administration device 1 preferably comprises a blocking device 12 for blocking or stopping the priming device 10 and/or for preventing the priming device 10 from being moved beyond a defined end position. Thus, the blocking device 12 preferably defines an end position for the priming device 10. The blocking device 12 is preferably arranged on an inner wall 16 of the housing 8 and/or formed in one piece with the inner wall 16. The blocking device 12 preferably forms a stop for the priming device 10. In particular, the blocking device 12 projects from the inner wall 16 of the housing 8 and/or forms an elevation of or on said inner wall 16.

The priming device 10 is preferably elongated and/or rod-like. At the end of the priming device 10 comprising the actuation section 11, the priming device 10 has preferably an enlarged cross section and/or diameter, in particular perpendicular to the longitudinal extension of the priming device 10 and/or axis L, so that the priming device 10 or actuation section 11 comprises a contact area 13 configured and/or arranged to contact the blocking device 12. The contact area 13 preferably extends transversely, in particular at least essentially perpendicularly, to the axis L and/or the longitudinal extension of the priming device 10.

In particular, the priming device 10 is (linearly) moveable into the housing 8 and/or administration device 1 and/or towards the syringe 2, in particular the piston 4, preferably until the contact area 13 contacts the blocking device 12, whereby further movement of the priming device 10 is blocked.

In FIG. 3, the priming device 10 is shown in an unactuated position. In FIG. 4, the priming device 10 has been moved from the unactuated position of FIG. 3 into the actuated or defined end position by moving the priming device 10 towards the outlet 7 and/or administration opening 15. This is indicated by the arrow in FIG. 4.

Thus, the piston 4 preferably cannot be moved beyond the predefined priming position by the priming device 10. In particular, it is not possible to reduce the volume of the chamber 6 to (at least essentially) zero by moving the piston 4 towards the outlet 7 by the priming device 10. In particular, actuation of the syringe 2 is performed by actuation of a different part other than the part which is actuated for priming, i.e., the priming device 10. Thus, priming cannot lead to reducing the chamber 6 to a volume smaller than intended. Consequently, it is prevented that a part of the dose of fluid F or drug to be administered to a patient is removed from the chamber 6 by or during priming

The syringe 2, piston 4, housing 8, blocking device 12, priming device 10 and/or contact area 13 are preferably dimensioned and/or arranged in such a way that the piston 4 is moved into the priming position when the priming device 10 is moved towards the piston 4 until the contact area 13 contacts the blocking device 12.

The actuation mechanism 9 is preferably configured for moving the syringe 2 or syringe body 3 relative to the housing 8.

Preferably, priming by the priming device 10 takes place or is performed before moving and/or actuating the syringe 2.

The actuation mechanism 9 comprises several parts which interact or work together, in particular to actuate the syringe 2 and/or move the syringe 2, in particular relative to the housing 8.

The actuation mechanism 9 is preferably configured for moving the syringe 2 relative to the housing 8 into the administration position. Said movement into the administration position can in particular be performed without actuating the syringe 2, i.e. without moving the piston 4 relative to the syringe body 3 while moving the syringe 2 by the actuation mechanism 9. This is in particular shown in FIGS. 4 and 5.

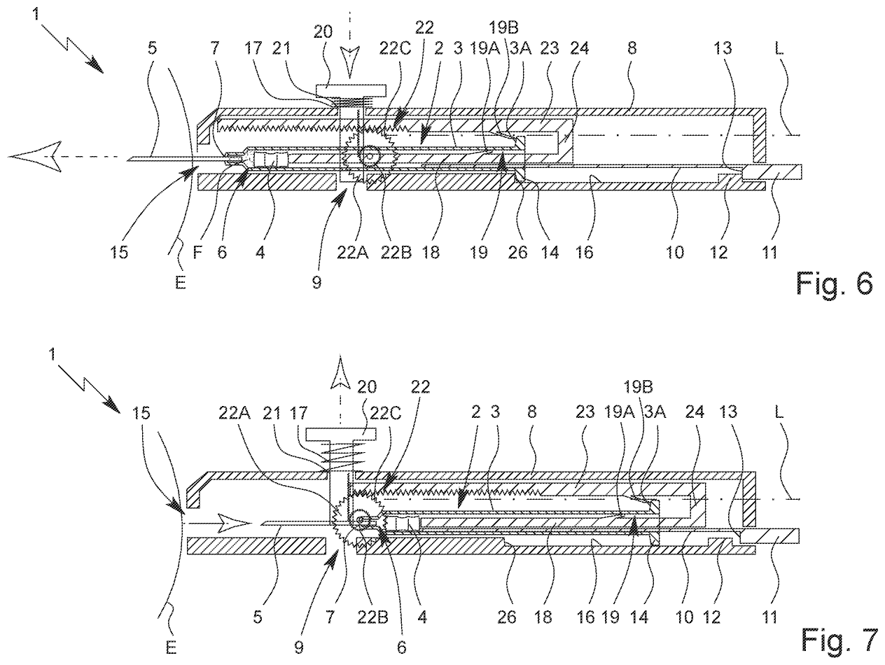

Further, the actuation mechanism 9 is preferably configured for actuating the syringe 2 or moving the piston 4 relative to the syringe body 3, preferably towards the outlet 7, in particular subsequent to moving the syringe 2 into the administration position. This is in particular shown in FIG. 6. The arrow in FIG. 5 indicates the movement of the syringe 2 from the rest position shown in FIG. 4 into the administration position shown in FIG. 5.

Further, the actuation mechanism 9 is preferably configured for positioning the syringe 2 and/or the injection needle 5 at least essentially completely inside the housing 8, in particular subsequent to actuating the syringe 2, for example by retracting the syringe 2 into the housing 8. Preferably, positioning the syringe 2 and/or injection needle 5 inside the housing 8 can be or is performed automatically. This is in particular shown in FIG. 7. The arrow in FIG. 6 indicates the movement of the syringe 2 from the administration position shown in FIG. 6 into the rest or retracted position shown in FIG. 7.

Thus, in a particularly preferred embodiment, the actuation mechanism 9 is configured to first move the syringe 2 into the administration position, to subsequently actuate the syringe 2 by moving the piston 4 towards the outlet 7 so that fluid F contained in the chamber 6 of the syringe 2 is dispensed, and to subsequently position the syringe 2 completely inside the housing 8 (again), for example by retracting the syringe 2 into the housing 8, so that the syringe 2 and/or the injection needle 5 do not jut out of the housing 8. Preferably, these steps are performed subsequently and/or automatically by one single actuation of the administration device 1 or actuation mechanism 9, as will be further detailed below. Consequently, a safe and easy handling of the administration device 1 can be achieved.

The actuation mechanism 9 preferably comprises a return means 17. The return means 17 is preferably configured and/or arranged to bias or force the syringe 2, in particular the syringe body 3, into the retracted or rest position.

The return means 17 is preferably a spring, particularly preferably a coil spring and/or a compression spring.

The actuation mechanism 9 preferably comprises a pushing element 18 configured for actuating the piston 4, in particular in addition to and/or independently from the priming device 10. Particularly, the pushing element 18 can be moved further towards the syringe 2 and/or the outlet 7 than the priming device 10. The pushing element 18 is preferably configured to move or push the piston 4 up to the axial end of the syringe 2 comprising the outlet 7, in particular so that a desired dose of fluid F, in particular all of the fluid contained in the chamber 6, can be dispensed through the outlet 7 by actuating, pushing or moving the piston 4 with the pushing element 18.The pushing element 18 is preferably separate from or physically isolated from and/or not rigidly connected to the piston 4.

The pushing element 18 is preferably rod-like and/or elongated and/or extends along and/or parallel to the axis L. Preferably, the pushing element 18 is partially arranged within the syringe body 3 and/or extends into the syringe body 3. Particularly preferably, the pushing element 18 is located in close proximity to the priming device 10 and/or extends at least essentially parallel to the priming device 10. The pushing element 18 is preferably located completely inside the housing 8. The pushing element 18 is preferably configured to move independently of the priming device 10.

The pushing element 18 preferably comprises a coupling device 19 for coupling the pushing element 18 to the syringe body 3.

Preferably, the pushing element 18 or coupling device 19 can be coupled with the syringe 2, in particular the syringe body 3, in two different coupling positions.

In a first coupling position, the pushing element 18 is preferably coupled to the syringe body 3 in such a way that the position of the syringe body 3 relative to the pushing element 18 is at least temporarily fixed, in particular so that the syringe 2 is moved out of the rest position and/or towards the administration position when the pushing element 18 is moved towards the administration opening 15. In the first coupling position, the pushing element 18 is preferably moveable relative to the priming device 10.

Preferably, the pushing element 18 can be coupled with the syringe body 3 in a second coupling position, in particular automatically and/or subsequent to the coupling in the first coupling position. Preferably, the coupling in the first coupling position is released and/or the pushing element 18 is moved relative to the syringe body 3 after coupling in the first position and/or before coupling in the second coupling position. It is particularly preferred that coupling in the second coupling position only occurs after the pushing element 18 has moved the piston 4 towards the outlet 7. Thus, coupling in the second coupling position preferably only occurs after dispensing fluid F from the syringe 2.

In other words, coupling in the first position preferably occurs in the rest position of the syringe 2 and/or during movement of the syringe 2 from the rest position into the administration position. Coupling in the second coupling position preferably only occurs when or after the syringe 2 has been moved into the administration position and/or during movement of the syringe 2 from the administration back into the rest position or retracted position.

The first coupling is depicted in FIGS. 3, 4 and 5. The second coupling is depicted in FIGS. 6 and 7.

The coupling device 19 preferably comprises or is formed by one or several flaps or arms 19A, 19B.

The pushing element 18 preferably comprises a first arm 19A which is preferably arranged laterally at the pushing element 18. In particular, the first arm 19A protrudes transversely or slanted from the pushing element 18 and/or is arranged transversely or slanted to the axis L and/or pushing element 18. The arm 19A is preferably built in one piece with the pushing element 18.

Coupling in the first coupling position is preferably effected by the first arm 19A. The diameter and/or cross section of the pushing element 18 is preferably enlarged by the first arm 19A. The diameter and/or cross section of the pushing element 18 with the first arm 19A is preferably larger than the inner diameter or cross section of the syringe body 3 so that the arm 19A contacts the axial end 14 of the syringe body 3.

The syringe body 3, pushing element 18 and arm 19A are preferably arranged in such a way that the arm 19A contacts the axial end 14 of the syringe 2 or syringe body 3 when the syringe 2 is in the rest position and/or the administration device 1, in particular the actuation element 20, is not actuated. This is in particular shown in FIGS. 3 and 4.

When the pushing element 18 is moved towards the administration opening 15, the syringe body 3 is preferably moved together with the pushing element 18 due to the coupling with the first arm 19A at the axial end 14. Thus, the pushing element 18 is preferably not moved relative to the syringe body 3 and/or the piston 4, so that the syringe 2 is not actuated by being moved into the administration position.

The administration device 1 preferably comprises a stop 26 for the syringe 2. In particular, the stop 26 is configured for stopping the syringe 2 in the administration position and/or defines the administration position. In particular, the stop 26 is configured to contact the collar 3A of the syringe 2.

The stop 26 is preferably located inside the housing 8. Preferably, the stop 26 is formed in one piece with the housing 8 or a part 8A, 8B thereof, in particular an inner wall thereof.

When the syringe 2 has reached the administration position, the pushing element 18 can preferably be further advanced towards the administration opening 15, in particular so that the piston 4 is actuated, in particular moved towards the outlet 7.

The coupling device 19 or first arm 19A is preferably flexible. In particular, the first arm 19A is configured to be deformed and/or swiveled towards the pushing element 18, thereby reducing the diameter and/or cross section of the pushing element 18, so that coupling between the arm 19A and the syringe body 3 is released and the pushing element 18 can be further advanced into the syringe body 3 in order to move or actuate the piston 4.

The first arm 19A is preferably deformed by (further) pushing the pushing element 18 towards the outlet 7 and/or (further) actuating or pressing down the actuation element 20 when the syringe 2 has (already) reached the administration position and/or when a further movement of the syringe 2 or syringe body 3 towards the administration opening 15 is blocked by the stop 26.

The coupling device 19 is preferably configured for coupling in the second coupling position only when or after the piston 4 has been moved towards the outlet 7 by the pushing element 18. The coupling device 19 preferably comprises a second arm 19B for coupling in the second position. The second arm 19B is preferably arranged on a rod 23.

The rod 23 is preferably rigidly connected to the pushing element 18, in particular by a connection part 24. The connection part 24 is preferably arranged between the pushing element 18 and the rod 23. The pushing element 18, rod 23 and connection part 24 preferably form a U-shape, in particular wherein the pushing element 18 and rod 23 are parallel to each other. The rod 23 is preferably positioned completely inside the housing 8 and/or outside the syringe 2 or syringe body 3.

The second arm 19B is preferably flexible. In particular, the second arm 19B is configured to be deformed and/or swiveled towards the rod 23, so that the rod 23 and/or second arm 19B can pass the collar 3A.

The second arm 19B is preferably configured to engage the collar 3A of the syringe 3, in particular on a side of the collar 3A facing the outlet 7. The second arm 19B is preferably elastic. The second arm 19B is preferably arranged at the rod 23 in such a way that the second arm 19B passes the collar 3A when the pushing element 18 pushes the piston 4 towards the outlet 7 and/or that the second arm 19B engages the collar 3A, in particular on a side facing the outlet 7, when the piston 4 has been moved sufficiently far towards the outlet 7.

When the pushing element 18 and/or rod 23 is moved away from the administration opening 15, in particular after administrating the fluid F, the syringe 2 or syringe body 3 is preferably moved together with the pushing element 18 and/or rod 23 due to the coupling in the second coupling position, in particular due to the engagement of the second arm 19B with the collar 3A.

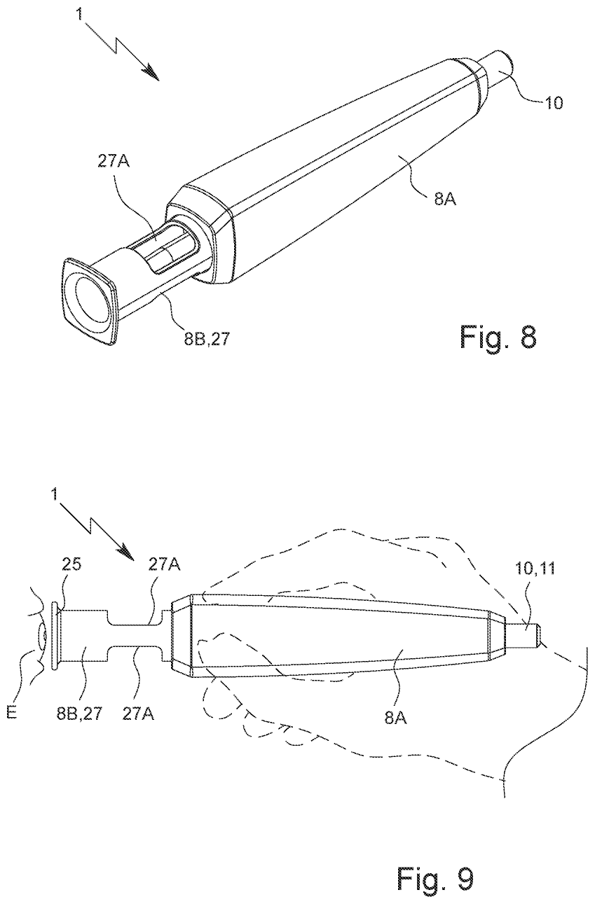

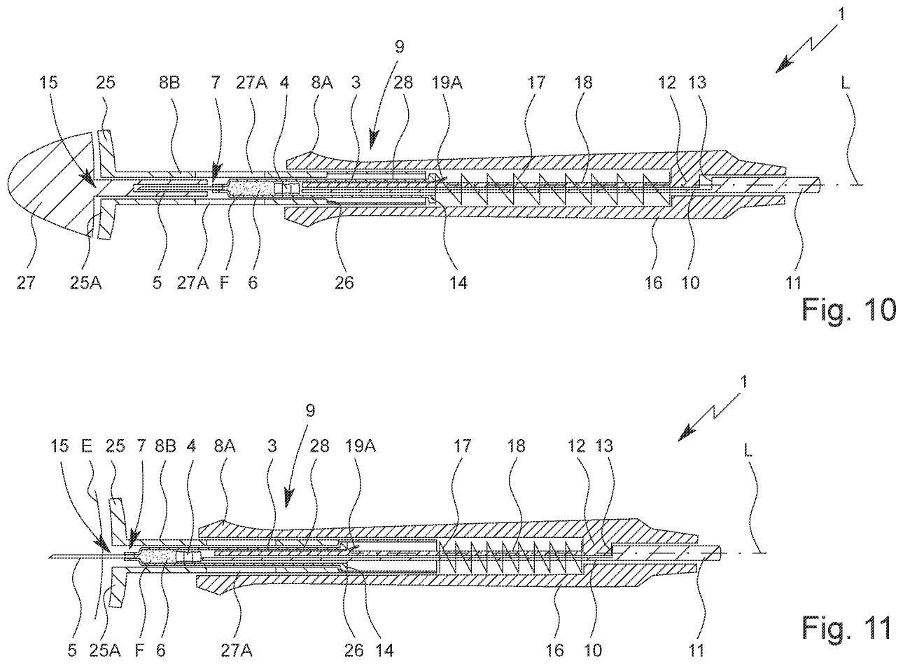

While the above explanations have predominantly been made with reference to the first embodiment shown in FIGS. 1 to 7, these explanations and features described also apply to the further embodiments shown in FIGS. 8 to 13 and are not restricted to the first embodiment.

In the following, some special features of the first embodiment will be described with reference in particular to FIGS. 1 to 7 before further embodiments are discussed.

The administration device 1 and/or actuation mechanism 9 preferably comprises an actuation element 20 configured for actuating the administration device 1, in particular the syringe 2 and/or the actuation mechanism 9.

The actuation element 20 is preferably configured or formed button-like and/or plunger-like.

The actuation element 20 is preferably arranged laterally at the administration device 1, in particular that an area or face of the actuation element which is configured to be actuated faces in a radial direction with respect to the axis L. Preferably, the actuation element 20 extends through an opening or recess 21 of the housing 8, the recess 21 preferably being arranged laterally at the housing 8. It is preferred that the actuation element 20 juts out of the housing 8.

The actuation element 20 is preferably arranged at or close to the axial end of the administration device 1 which comprises or forms the administration opening 15. The actuation element 20 is preferably arranged at or close to the axial end of the administration device 1 which is opposite to the (axial) end comprising the priming device 10 and/or the actuation section 11 thereof. Preferably, the actuation element 20 is arranged closer to the end comprising the administration opening 15 than to the end comprising the priming device 10.

The actuation element 20 preferably extends transversely, in particular at least essentially perpendicularly, to the axis L and/or the direction of movement of the pushing element 18 and/or the direction of movement of the priming device 10.

Preferably, the actuation element 20 is moveable in a direction transverse, in particular perpendicular to the axis L and/or the direction of movement of the pushing element 18 and/or the direction of movement of the priming device 10.

Preferably, the actuation element 20 is configured to be actuated manually and/or directly by a user of the administration device 1, in particular by an index finger of the user.

As shown in FIG. 2, the administration device 1 and/or actuation element 20 is preferably arranged and/or configured for one-handed actuation, in particular in such a way that the administration device 1 can be held like a pen and/or between the thumb and the index finger and/or middle finger of a user of the administration device 1, in particular so that the actuation element 20 can be actuated, in particular pressed down, with the index finger while holding the administration device 1. Preferably, it is not necessary for the user to change his grip in order to actuate the administration device 1 and/or actuation element 20, in particular during and/or after piercing the body, in particular eye, of a patient with the needle 5. This makes using the administration device 1 easier and safer. Preferably, wobbling or wiggling of the administration device 1 during usage is thus prevented.

The administration device 1 is preferably constructed in such a way that actuation of the administration device 1 and/or administration of the fluid F is performed in a different manner than by pressing on an actuation device such as a piston or plunger of a syringe located at an axial end of the administration device 1 or syringe 2. Preferably, the administration device 1 does not comprise a device for actuating the syringe 2 which is located and/or configured to be actuated at an axial end of the administration device 1 opposite the axial end comprising the outlet 7 and/or the administration opening 15.

The actuation element 20 is preferably biased and/or forced into an unactuated position, in particular by the return means 17. This is in particular displayed in FIGS. 3 to 7.

The spring or return means 17 is preferably arranged outside the housing 8 and/or between the housing 8 and the actuation element 20, in particular transversely or perpendicularly to the axis L and/or the direction of movement of the priming device 10 and/or the direction of movement of the pushing element 18.

The return means 17 is preferably configured to be compressed upon actuation of the actuation element 20.

The pushing element 18 is preferably moveable or actuatable by an actuation of the actuation element 20, in particular by pressing down the actuation element 20, i.e. by moving the actuation element 20 towards the housing 8 and/or in a direction perpendicular to the axis L.

The actuation element 2 is preferably provided in addition and/or separately from the priming device 10. The actuation element 20 is preferably mechanically and/or functionally decoupled from the priming device 10, in particular so that the actuation element 20 and priming 10 can be actuated independently and/or work or move independently form each other.

The actuation element 20 and/or priming device 10 can be configured so that actuation or pressing down of the actuation element 20 is blocked, in particular by the priming device 10, as long as priming has not been performed or the priming device 10 has not been advanced to the priming position.

The actuation mechanism 9 preferably comprises a gear or transmission 22. The transmission 22 is preferably configured to transmit or convert an actuation or movement of the actuation element 20 to the pushing element 18 or a movement thereof. In particular, the transmission 22 is configured to convert a movement of the actuation element 20 to a movement of the pushing element 18 which is transverse, in particular at least essentially perpendicular, to the direction of movement of the actuation element 20.

Further, the transmission 22 preferably comprises or forms a gear train. The gear ratio, in particular the ratio between the distance of movement of the actuation element 20 and the distance of movement of the pushing element 18 and/or rod 23, is preferably greater than 1:3, particularly greater than 1:5, and/or smaller than 1:20, particularly smaller than 1:10.

The transmission 22 preferably comprises a gearwheel 22A. Particularly preferably, the gearwheel 22A is a double gearwheel, i.e. a gearwheel comprising two different annular gears or toothed rims 22B, 22C. The two annular gears or toothed rims 22B, 22C preferably have different diameters, in particular wherein the diameter of the first rim 22B, hereinafter denoted as inner rim 22B, is smaller than the diameter of the second toothed rim 22C, hereinafter denoted as outer rim 22C.

The actuation element 20 preferably comprises one or more toothed racks or rods. In particular, the gearwheel 22A and the actuation element 20 together form one or more rack and pinions.

The actuation element 20 preferably comprises or forms one of said toothed rods. In particular, the actuation element 20 and the inner toothed rim 22B of the gear wheel 22A together form a first rack and pinion.

Preferably, the rod 23 comprises or forms a second of said toothed rod. In particular, the rod 23 and the outer toothed rim 22C of the gear wheel 22A form a second rack and pinion.

In this way, the relatively small distance of movement of the actuation element 20 is converted to a much larger distance of movement of the pushing element 18. This is advantageous for an easy handling of the administration device 1.

Moving the syringe 2 into the administration position and/or actuating the syringe 2, in particular dispensing the fluid F, can preferably be performed by a single and/or continuous actuation of the actuation element 20, in particular by pressing down the actuation element 20 (towards the housing 8 and/or in a direction transverse to the axis L). The syringe 2 is preferably automatically retracted into the housing 8 or moved (back) into the retracted or rest position, in particular after administrating the fluid F and/or actuating the actuation element 20.

In the sense of the present invention, “retracting” the syringe 2 into the housing 8 preferably means a movement of the syringe 2 or syringe body 3 relative to the housing 8 or a part 8A, 8B thereof so that the syringe 2 is positioned inside the housing 8 when retracting is finished. In particular, the syringe 2 and/or injection needle 5 does not jut out of the housing 8 in the retracted position. In one embodiment, retracting the syringe 2 can be performed by moving the syringe 2 relative to the housing 8. In another embodiment, however, it is also possible that the housing parts 8A, 8B are moved relative to each other and/or at least one of the housing parts 8A, 8B is moved relative to the syringe 2 or syringe body 3 so that the syringe 2 gets surrounded and/or covered by the housing 8 by said relative movement.

In particular, the term “retracting the syringe 2” refers to a movement of the syringe 2 from the administration position into the retracted or rest position.

The return means 17 is preferably configured for (automatically) positioning the syringe 2 in the housing 8 and/or retracting the syringe 2 into the housing 8. The return means 17 is preferably configured to (automatically) move the syringe 2 from the administration position to the retracted or rest position. In particular, the return means 17 is configured to move the syringe 2 by returning the actuation element 20 into a starting position in which the actuation element 20 is/was positioned before being actuated or pressed down. The movement of the actuation element 20 is preferably transferred to a movement of the syringe 2 into the retracted or rest position by the actuation mechanism 9, in particular the transmission 22, pushing element 18, coupling device 19 and/or rod 23.

In the following, in particular the administration device 1 according to a second embodiment which is shown in FIGS. 7 to 12 will be described. As explained above, the second embodiment shares a plurality of features with the first embodiment. Thus, predominantly differences of the second embodiment compared to the first embodiment will be discussed in the following. Thus, if not indicated otherwise, the above explanations related to the first embodiment preferably also hold for the second embodiment.

According to the second embodiment, the administration device 1 or housing 8 preferably comprises two housing parts 8A and 8B, in the following differentiated by the terms “first housing part 8A” and “second housing part 8B”.

The housing parts 8A, 8B are preferably arranged coaxially to each other and/or the axis L.

The housing parts 8A, 8B are preferably moveable relative to each other, in particular linearly and/or along the axis L. The housing parts 8A, 8B are preferably moveable relative to each other in a telescopic fashion.

Preferably, the second housing part 8B extends at least partially into the first housing part 8A and/or the first housing part 8A surrounds the second housing part 8B at least partially, or vice versa.

The administration device 1, in particular the housing 8, particularly preferred the second housing part 8B, preferably comprises or forms a cover or shield 27 for the injection needle 5, the housing part 8B preferably surrounding the injection needle 5 at least radially. However, the shield 27 can also be a part which is detachable from the housing 8 or first housing part 8A.

The shield 27 preferably comprises one or more openings or recesses 27A, in particular when the injection needle 5 is connectable to and/or detachable from the administration device 1 and/or syringe 2, for example by a Luer connection.

The recess/recesses 27A is/are preferably arranged laterally on the shield 27. Preferably, two recesses 27A are provided, in particular wherein the recesses 27A are arranged on radially opposing sides of the shield 27.

The injection needle 5 and/or the inside of the shield is preferably accessible by the recess/es 27A. The recesses 27A are preferably configured to allow and/or facilitate connecting the injection needle 5 to the syringe 2, in particular by a Luer connection, and/or removing or detaching the injection needle 5 from the syringe 2. Further, inspection of the injection needle 5 is made possible by the recess/es 27A.

The administration device 1 preferably comprises an alignment device 25 for aligning the administration device 1 to an eye E of a patient.

Preferably, the second housing part 8B forms or comprises the alignment device 25.

The alignment device 25 is preferably arranged at the axial end of the housing 8 or housing part 8B comprising the administration opening 15.

The alignment device 25 preferably comprises or forms an alignment area 25A. The alignment device 25 and/or alignment area 25A is preferably configured for direct contact with an eye E of a patient. Preferably, the alignment area 25A extends transversely to the longitudinal extension of the second housing part 8B and/or axis L. The alignment area 25A is preferably arranged at an axial end of the alignment device 25.

The alignment area 25A is preferably at least substantially flat, planar and/or laminar

In particular, the alignment device 25 and/or alignment area 25A is curved and/or comprises a (slight) curvature and/or an at least essentially spherical shape, thus particularly being adapted to the shape of the eye E.

The alignment device 25 is preferably elongated, tube-like, sleeve-shaped and/or at least essentially cylindrical. In particular, the alignment device 25 surrounds the needle 5, at least partially.

The actuation mechanism 9 of the administration device 1 according to the second embodiment is in principle very similar and/or partly identical to the actuation mechanism 9 of the administration device 1 according to the first embodiment. In particular, the above explanations concerning the priming device 10 and the actuation of the syringe 2 by the pushing element 18, in particular the steps of first moving the syringe 2 to an actuation position, then actuating the syringe 2 and subsequently positioning the syringe 2 within the housing 8 again or retracting the syringe 2 also hold for the second embodiment.

In contrast to the first embodiment, the administration device 1 according to the second embodiment preferably does not comprise a button-like actuation element.

The administration device 1 and/or syringe 2 according to the second embodiment is preferably configured to be actuated by moving the housing parts 8A, 8B relative to each other, in particular by a linear movement.

Further, in the second embodiment, the spring or return means 17 is arranged inside the housing 8 and/or between the first housing part 8A and the second housing part 8B. The return means 17 is preferably arranged coaxially to the longitudinal extension of the housing 8 or the housing parts 8A, 8B and/or the axis L.

Preferably, the return means 17 is configured or arranged to force the first and second housing parts 8A, 8B apart and/or into a rest position.

The pushing element 18 of the second embodiment is preferably formed in one piece with the housing 8, in particular the first housing part 8A, and/or is not moveable relative to the first housing part 8A.

The housing 8, in particular the first housing part 8A, preferably forms a part of the actuation mechanism 9, in particular by comprising or forming the pushing element 18.

The pushing element 18 and/or priming device 10 are preferably arranged coaxially to the return means 17 and/or extend into or through the return means 17. In particular, the return means 17 surrounds the pushing element 18 and/or the priming device 10.

The actuation mechanism 9 according to the second embodiment does preferably not comprise a transmission 22.

The syringe 2 is preferably configured to be moved into the administration position and/or actuated by an in particular linear movement of the first housing part 8A relative to the second housing part 8B. By this movement, the return means 17 is preferably being compressed.

The stop 26 is preferably arranged on the second housing part 8B, in particular an inner wall thereof.

The pushing element 18 can be or is preferably coupled to the syringe 2 by the coupling device 19, in particular an arm 19A, 19B thereof, as explained above in connection with the first embodiment. Preferably, the position of the syringe 2 relative to the first housing part 8A and/or the pushing element 18 is fixed by the coupling, at least until the syringe 2 has reached the administration position.

In other words, the syringe 2 is preferably configured to be moved relative to the second housing part 8B, in particular from the rest position into the administration position, when the first housing part 8A is moved relative to the second housing part 8B, in particular towards the administration opening 15.

When the syringe 2 has reached the administration position, the syringe 2 can preferably be actuated and/or fluid F can be administered by further movement of the first housing part 8A relative to the second housing part 8B. By said further movement, the coupling of the coupling device 19 or arm 19A with the syringe body 3 is preferably released, so that the pushing element 18 is further moved towards the piston 4 and thus actuates the piston 4 or syringe 2.

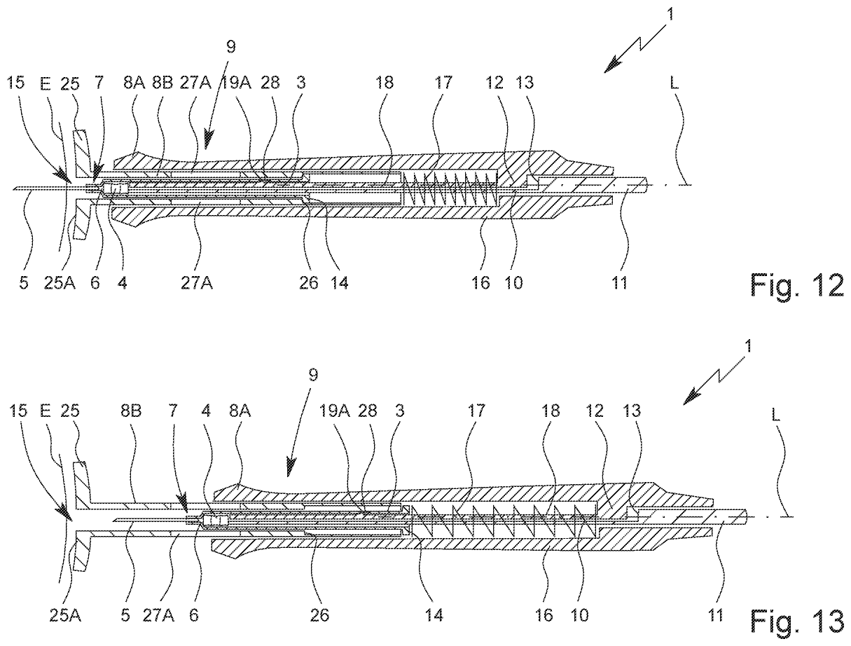

Upon actuation of the piston 4, in particular moving the piston 4 towards the outlet 7, the first housing part 8A is preferably moved relative to the syringe 2 and/or the second housing part 8B, in particular so that the syringe 2 and/or the second housing part 8B are moved into or towards the first housing part 8A. This can be seen in particular by comparison of FIGS. 11 and 12.

Subsequently, the syringe 2, in particular the injection needle 5, can preferably be positioned completely inside and/or retracted into the housing 8, in particular automatically.

The coupling device 19 according to the second embodiment preferably comprises only or exactly one coupling arm 19A. The coupling arm 19A can preferably be coupled with the syringe body 3 in two different coupling positions.

The syringe body 3 preferably comprises a recess 28 or the like configured for coupling with the arm 19A in a second coupling position. In a preferred embodiment, the arm 19A is configured to engage with the recess 28 of the syringe body 3 and thereby couple to the syringe body 3 in the second coupling position when the piston 4 is pushed towards the outlet 7 by the pushing element 18. In particular, the arm 19A is elastic so that it automatically engages the recess 28 when reaching the position thereof.

The coupling of the arm 19A in the second coupling position in particular enables the retracting of the syringe 2 into the housing 8.

The solution with the coupling device 19 comprising only one arm 19A which is configured to engage with the syringe body 3 in two different positions can preferably also be realized in the first embodiment of the administration device.

The return means 17 is preferably configured to (automatically) position the syringe 2 inside the housing 8 or to retract the syringe 2 into the housing 8. In particular, the administration device 1 is configured to retract the syringe 2 by moving the second housing part 8B relative to the first housing part 8A, in particular by moving the housing parts 8A, 8B apart by the (compressed) spring or return means 17, particularly so that the second housing part 8B is pushed out of the first housing part 8A, whereby the second housing part 8B covers the syringe 2 and/or injection needle 5. In particular, the second housing part 8B is moved relative to the syringe 2 and/or the syringe 2 is not moved relative to the first housing part 8A upon retracting the syringe 2.

The administration device 1 is in particular configured to perform the method described in the following.

A method for using or actuating the administration device 1 and/or for dispensing or administrating a fluid F from/with the administration device 1 and/or for administering or injecting a drug to a patient, in particular directly to the body of the patient or a part thereof, preferably comprises one or more of the following steps:

Preferably, first the shield 27 is detached from the administration device 1.

The priming device 10 is preferably advanced to a defined end position, in particular by pushing the priming device 10 towards the administration opening 15, thereby preferably moving the priming device parallel to the axis L. By moving the priming device, the piston 4 is preferably moved or pushed into the priming position.

It is possible that priming by the priming device 10 is performed before removing the shield 27.

The administration device 1 is preferably positioned in a desired position relative to the body of a patient to which a drug or fluid F is to be administered, for example placed on or above a vein into which a drug is to be injected or placed on the eye E into which the drug is to be injected.

Additionally, it is possible that the eye E of the patient is anaesthetized before positioning the administration device 1 relative to the eye E.

Positioning the administration device 1 can be supported or facilitated by the alignment device 25.

The administration device 1 is preferably actuated after priming or actuating the priming device 10. According to a preferred aspect, the administration device 1 is not actuatable before priming and/or only actuatable after priming or actuating the priming device.

Actuation of the administration device 1 is preferably performed by actuating the actuating device 20 and/or moving the housing parts 8A, 8B relative to another.

The fluid F or drug contained in the administration device 1 or syringe 2 is preferably administered to the patient. This is preferably performed by actuating the administration device 1, for example by actuating or pressing down the actuation element 20 and/or by moving the housing parts 8A, 8B relative to one another, preferably so that the syringe 2 is first moved into the administration position and then actuated, to that the fluid F is expelled from the syringe 2 and/or administered to the patient.

After administering the fluid F, the injection needle 5 is preferably retracted into the administration device 1, in particular the housing 8. This is preferably performed automatically, in particular by releasing the actuation element 20 and/or the housing parts 8A, 8B for retracting the syringe 2. In particular, a force used for actuating the actuation element 20 and/or moving the housing parts 8A, 8B relative to one another is reduced or removed, for example by taking away a finger used for pressing down the actuation element 20.

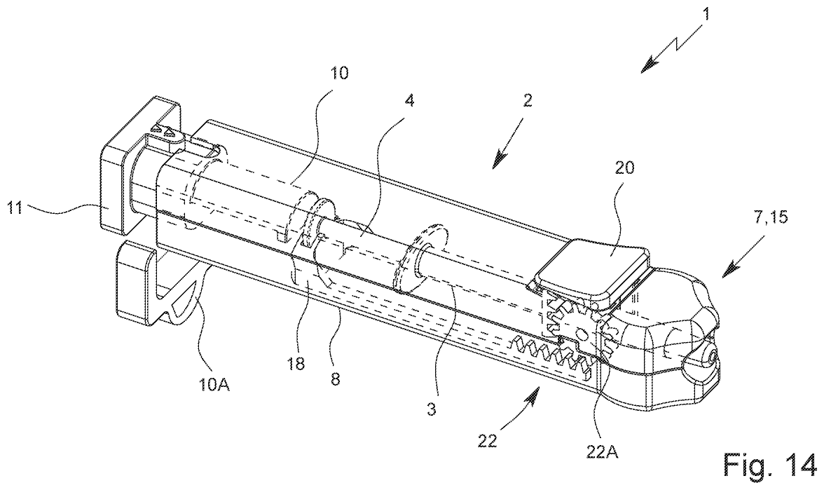

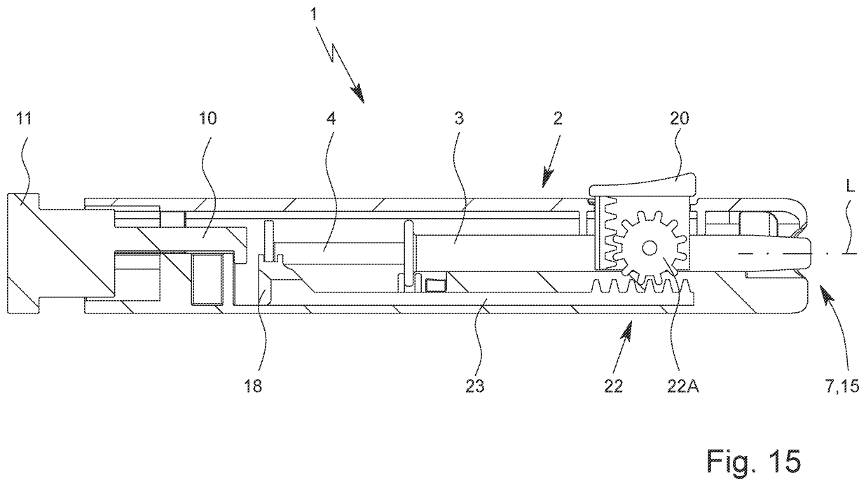

In the following, a particularly preferred embodiment of the present invention will be described. The preferred embodiment is displayed in FIGS. 14 and 15.

The third embodiment of the administration device 1 is similar to the first embodiment. Thus, only differences with respect to the first embodiment will be described in the following and the above explanations, in particular those in connection with the first embodiment, preferably also apply to the third embodiment.

The administration device 1 according to the third embodiment preferably comprises a locking device 10A for preventing (unintended) priming, in particular for preventing movement of the priming device 10 towards the syringe 2. The locking device 10A is preferably configured for (mechanically) locking the priming device 10. Preferably, the locking device 10A forms a stop for the priming device 10.

The locking device 10A is preferably arranged or arrangable between the priming device 10, in particular the actuation section 11, and the housing 8 and/or a part thereof.

The locking device 10A is preferably configured to encompass the priming device 10 at least partly. In particular, the locking device 10A has a U-shape.

The locking device 10A is preferably removable from the priming device 10 and/or administration device 1, in particular in a direction transverse, in particular perpendicular, to the axis L.

The administration device 1 according to the first embodiment can also comprise the locking device 10A.

In the third embodiment, the position of the syringe 2, in particular the syringe body 3, in the housing 8 is preferably fixed and/or the syringe 2, in particular the syringe body 3, is not moveable relative to the housing 8.

The pushing element 18 is preferably formed by a plate-like and/or flat element configured to act on the piston 4. As an alternative or in addition, the pushing element 18 or a section thereof can be configured to encompass or grip, at least partly, the piston 4, in particular a radially extending plate arranged at an axial end of the piston 4. This is in particular shown in FIG. 15.

The administration device 1 according to the third embodiment is preferably configured so that only the piston 4 of the syringe 2 is moved by actuating the actuation element 20 and/or so that the body 3 of the syringe 3 is not moved or not moveable by actuating the actuation element 20. The gear ratio of the transmission 22 is preferably at least essentially 1:1. The gearwheel 22A preferably comprises only or exactly one toothed rim.

The pushing element 18 is preferably directly connected to the rod 23 and/or extends transversely, in particular perpendicularly, to the rod 23.

The administration device 1 according to the third embodiment can comprise the return means 17 (not shown in FIGS. 14 and 15) for returning the actuation element 20 into the unactuated position after actuating, as described above. Preferably, the actuation element 20 and/or return means 17 is, however, not coupled to the syringe 2 or syringe body 3 and/or is not configured to move the syringe 2 relative to the housing 8. Thus, the return means 17 is preferably only configured for returning the actuation element 20 into the unactuated position after actuating.

The administration device 1 according to the third embodiment preferably comprises the needle shield 27 as described above in connection with the second embodiment.

Individual aspects and features of the present invention may be implemented independently from one another, but also in any desired combination and/or order.

1. Administration device (1), in particular intravitreal administration device (1), for administration of a fluid (F), the administration device (1) comprising a syringe (2) and an actuation mechanism (9) for actuating the syringe (2), characterized
in that the administration device (1) comprises a priming device (10) configured to act on a piston (4) of the syringe (2) and a blocking device (12) for preventing the priming device (10) from moving the piston (4) to an end of the syringe (2), and/or
in that the administration device (1) comprises a priming device (10) in addition to the actuation mechanism (9).

2. Adminstration device according to claim 1, characterized in that the priming device (10) is configured and/or arranged to actuate the piston (4) and/or to move the piston (4) relative to the syringe body (3).

3. Adminstration device according to claim 1 or 2, characterized in that the priming device (10) is configured to move or push the piston (4) towards an outlet (7) of the syringe (2).

4. Adminstration device according to one of the preceding claims, characterized in that the priming device (10) is physically isolated from the piston (4).

5. Adminstration device according to one of the preceding claims, characterized in that the priming device (10) comprises an actuation section (11) which is arranged outside the housing (8).

6. Adminstration device according to one of the preceding claims, characterized in that the priming device (10) is elongated and/or rod-like.

7. Adminstration device according to one of the preceding claims, characterized in that the blocking device (12) defines an end position for the priming device (10).

8. Adminstration device according to one of the preceding claims, characterized in that the blocking device (12) forms a stop for the priming device (10).

9. Adminstration device according to one of the preceding claims, characterized in that the blocking device (12) projects from an inner wall (16) of the housing (8) and/or forms an elevation of or on said inner wall (16).

10. Adminstration device according to one of the preceding claims, characterized in that the priming device (10) is separate and/or functionally decoupled from the actuation mechanism (9).

11. Adminstration device according to one of the preceding claims, characterized in the actuation mechanism (9) comprises a pushing element (18) configured for actuating the piston (4).

12. Adminstration device according to claim 11, characterized in that the administration device (1) comprises the pushing element (18) in addition to the priming device (10).

13. Adminstration device according to claim 11 or 12, characterized in that the pushing element (18) is configured for actuating the piston (4) independently from the priming device (10).

14. Adminstration device according to one of claims 11 to 13, characterized in that the pushing element (18) is located in close proximity to the priming device (10).

15. Adminstration device according to one of claims 11 to 14, characterized that the pushing element (18) extends at least essentially parallel to the priming device (10).

16. Administration device (1) for administration of a fluid (F), preferably the administration device (1) being configured according to one of the preceding claims, the administration device (1) comprising a housing (8), a syringe (2) and an actuation mechanism (9) for actuating the syringe (2),
characterized
in that the syringe (2) and/or an injection needle (5) attached thereto is or can be positioned completely inside the housing (8), and/or
in that the actuation mechanism (9) is configured for moving the syringe (2) relative to the housing (8).

17. Administration device according to claim 16, characterized in that the actuation mechanism (9) is configured for moving the syringe (2) relative to the housing (8) into an administration position.

18. Administration device according to claim 16 or 17, characterized in that the actuation mechanism (9) is configured for moving the syringe (2) without actuating the syringe (2).

19. Administration device according to one of claims 16 to 18, characterized in that the actuation mechanism (9) is configured for actuating the syringe (2) by moving a piston (4) of the syringe (2) subsequent to moving the syringe (2) into the administration position.

20. Administration device according to one of claims 16 to 19, characterized in that the actuation mechanism (9) is configured for positioning the syringe (2) and/or injection needle (5) completely inside the housing (8) subsequent to actuating the syringe (2).

21. Administration device according to one of claims 16 to 19, characterized in that the actuation mechanism (9) is configured for automatically positioning the syringe (2) and/or injection needle (5) completely inside the housing (8) subsequent to actuating the syringe (2).

22. Administration device according to one of claims 16 to 21, characterized in that the actuation mechanism (9) comprises a spring for moving the syringe (2) and the housing (8) or a part (8A, 8B) thereof relative to each other.

23. Administration device according to claim 22, characterized in that the spring is arranged within the housing (8).

24. Administration device according to one of claims 16 to 23, characterized in that the actuation mechanism (9) comprises a pushing element (18) for actuating a piston (4) of the syringe (2), the pushing element (18) comprising a coupling device (19) for coupling to a body (3) of the syringe (2).

25. Administration device according to one of claims 16 to 24, characterized in that the syringe (2) is retractable into the housing (8) after administration or being positioned in an administration position.

26. Administration device according to claim 25, characterized in that the syringe (2) is retractable automatically.

27. Administration device according to one of claims 16 to 26, characterized in that the housing (8) comprises two housing parts (8A, 8B) which are moveable relative to each other and/or the syringe (2).

28. Administration device according to claims 22 and 27, characterized in that the spring is arranged between the two housing parts (8A, 8B).

29. Administration device according to claim 28, characterized in that the spring is configured to move the housing parts (8A, 8B) apart.

30. Administration device according to one of claims 27 to 29, characterized in that the administration device (1) is configured so that during actuation of a piston (4) of the syringe (2), a first one of the housing parts (8A,8B) moves relative to the syringe body (3) and a second one of the housing parts (8A, 8B), while the syringe body (3) and the first housing part (8A) are not moved relative to each other.

31. Administration device according to one of claims 27 to 30, characterized in that a pushing element (18) for moving the piston (4) is rigidly connected to the first housing part (8A).

32. Administration device according to one of the preceding claims, characterized in that the administration device (1) is configured for intravitreal administration of the fluid (F).

33. Adminstration device according to one of the preceding claims, characterized in that the syringe (2) is a pre-filled syringe.

34. Administration device according to one of the preceding claims, characterized in that the syringe (2) comprises or is connectable to an injection needle (5), in particular by a Luer connection.

35. Administration device according to one of the preceding claims, characterized in that the actuation mechanism (9) is configured for actuating the syringe (2) by moving a piston (4) of the syringe (2).

36. Administration device according to one of the preceding claims, characterized in that the actuation mechanism (9) comprises an actuation element (20).

37. Administration device according to claim 36, characterized in that the actuation element (20) is button-like.

38. Administration device according to claim 36 or 37, characterized in that the actuation element (20) is configured for manual actuation independently of the priming device (10).

39. Administration device according to one of claims 36 to 38, characterized in that the actuation element (20) is configured to be actuated in a direction transverse, in particular perpendicular, to a longitudinal extension of the syringe (2).

40. Administration device according to one of claims 36 to 39, characterized in that the actuation element (20) is configured to be actuated in a direction transverse, in particular perpendicular, to a direction of movement of the priming device (10).

41. Administration device according to one of claims 36 to 40, characterized in that the actuation element (20) is arranged laterally at the housing (8).

42. Administration device according to one of claims 36 to 41, characterized in that the actuation element (20) is arranged at or close to an axial end of the administration device (1) comprising an administration opening (15).

43. Administration device according to one of claims 36 to 42, characterized in that the actuation element (20) is arranged close to an axial end of the administration device (1) which is opposite to an axial end comprising the priming device (10) or an actuation section (11) thereof.

44. Administration device according to one of the preceding claims, characterized in that the actuation mechanism (9) comprises a transmission (22) for moving the syringe (2).

45. Administration device according to claims 25 and 44, characterized in that the syringe (2) is retractable by the transmission (22).

46. Administration device according to claim 43 or 44, characterized in that the transmission (22) is driven by the actuation element (20).

47. Administration device according to one of the preceding claims, characterized in that the administration device (1) comprises an alignment device (25) configured to align the administration device (1) to an eye (E) of a patient.

48. Administration device according to claim 47, characterized in that the alignment device (25) comprises an alignment area (25A) configured for direct contact with an eye (E) of a patient.

49. Administration device according to claim 47 or 48, characterized in that the alignment area (25A) is at least substantially laminar.

50. Administration device according to one of claims 47 to 49, characterized in that the alignment area (25A) is curved.

51. Administration device according to one of claims 47 to 50, characterized in that the alignment device (25) comprises or forms an alignment area (25A) extending transversely to a longitudinal extension of the housing (8) or a part (8A, 8B) thereof and/or an axis (L) of the administration device (8).

52. Administration device according to one of claims 47 to 51, characterized in that the housing (8) or a part (8A, 8B) thereof comprises or forms the alignment device (25).

53. Use of an administration device (1) according to one of the preceding claims for intravitreal injection or injection directly into an eye (E) of a fluid (F), in particular a medicament.
